(19) United States
(12) Patent Application Publication
(10) Pub. No.: US 2018/0370871 A1
(43) Pub. Date: Dec. 27, 2018

(54) PROCESS AND APPARATUS FOR HYDROISOMERIZING A HYDROPROCESSED LIQUID STREAM

(71) Applicant: UOP LLC, Des Plaines, IL (US)

(72) Inventors: Simerjeet Singh, Windsor (GB); Michael A. Spivey, Kuala Lumpur (MY)

(21) Appl. No.: 15/630,414

(22) Filed: Jun. 22, 2017

(51) Int. Cl. C07C 5/13; B01D 3/14; B01D 53/60; C10G 45/08

(52) U.S. Cl. CPC C07C 5/13; B01D 3/14; B01J 23/74; C10G 45/08; B01D 53/60

(57) ABSTRACT

A hydroisomerization reactor is moved to a low pressure section downstream of a high pressure hydroprocessing unit. The hydroisomerization reactor can be easily taken off line during the warmer months when cold flow property specifications are less stringent. The hydroisomerization reactor is also operated at lower pressure than the hydroprocessing reactor requiring less capital and operating expense.

### FIELD

The field is the hydroisomerization of a hydroprocessed liquid stream.

### BACKGROUND

Hydroprocessing can include processes which convert hydrocarbons in the presence of hydroprocessing catalyst and hydrogen to more valuable products.

Hydrotreating is a hydroprocessing process used to remove heteroatoms such as sulfur and nitrogen from hydrocarbon streams to meet fuel specifications and to saturate olefinic or aromatic compounds. Hydrotreating can be performed at high or low pressures, but is typically operated at lower pressure than hydrocracking. Hydrocracking is a hydroprocessing process in which hydrocarbons crack in the presence of hydrogen and hydrocracking catalyst to lower molecular weight hydrocarbons. Hydroisomerization or dewaxing is a hydroprocessing process that increases the alkyl branching on a hydrocarbon backbone in the presence of hydrogen and hydroisomerization catalyst to improve cold flow properties of the hydrocarbon.

Diesel fuel streams must meet cold flow property specifications particularly for winter fuel use. One cold flow property is “pour point” which is the temperature at which a hydrocarbon stream becomes semi-solid and loses its flow characteristics. A high pour point is generally associated with a higher normal paraffin content or a normal paraffin content comprising higher carbon number. Another cold flow property is “cloud point” which is the temperature below which wax in the hydrocarbon stream begins to form a cloudy appearance. The “cold filter plugging point” of diesel fuel is the temperature at which the presence of solidified waxes clogs fuel filters and injectors in engines. The wax also can accumulate on cold surfaces such as on a pipeline or heat exchanger tube and form an emulsion with water.

When hydrocracking gas oil, cold flow property specifications for diesel product can limit the obtainable diesel yield by requiring a lower diesel cut point. It is desirable to decrease the product diesel cold flow property temperature values without reducing the diesel cut point to preserve more diesel yield. This can be accomplished by adding a hydroisomerization unit to decrease cold flow property temperature values without decreasing the diesel cut point. Hydrotreated diesel may also be hydroisomerized to improve its cold flow properties.

Cold flow properties are typically only a concern in the winter months when ambient temperatures are cooler. Consequently, the hydroisomerization reactor may be shut down in months outside of winter because cold flow property improvement is not necessary. Locating the hydroisomerization catalyst in the reactor with the other hydroprocessing catalyst may result in compromising or reducing hydroprocessing catalyst performance. Locating the hydroisomerization catalyst in a separate reactor in the high pressure loop requires taking the hydroisomerization reactor off line during summer months. However, the procedure for shut down is complicated, and shut down requires high pressure isolation valves and a complicated depressurization circuit.

There is a continuing need, therefore, for improved methods and apparatuses for hydroprocessing and hydroisomerizing hydrocarbon streams.

### BRIEF SUMMARY

A hydroisomerization reactor is located in a low pressure section downstream of a high pressure hydroprocessing unit. The hydroisomerization reactor can be easily taken off line during the warmer months when cold flow property specifications are less stringent. The hydroisomerization reactor is also operated at lower pressure than the hydroprocessing reactor requiring less capital and operating expense.

### BRIEF DESCRIPTION OF THE DRAWINGS

FIG. 1 is a schematic drawing of a hydrotreating unit with a hydroisomerization reactor downstream of a fractionation section.

FIG. 2 is a schematic drawing of a hydrocracking unit with a hydroisomerization reactor downstream of a fractionation section.

### DEFINITIONS

The term “communication” means that material flow is operatively permitted between enumerated components.

The term “downstream communication” means that at least a portion of material flowing to the subject in downstream communication may operatively flow from the object with which it communicates.

The term “upstream communication” means that at least a portion of the material flowing from the subject in upstream communication may operatively flow to the object with which it communicates.

The term “direct communication” means that flow from the upstream component enters the downstream component without undergoing a compositional change due to physical fractionation or chemical conversion.

The term “bypass” means that the object is out of downstream communication with a bypassing subject at least to the extent of bypassing.

As used herein, the term “a component-rich stream” means that the rich stream coming out of a vessel has a greater concentration of the component than the feed to the vessel.

As used herein, the term “a component-lean stream” means that the lean stream coming out of a vessel has a smaller concentration of the component than the feed to the vessel.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Absorber and scrubbing columns do not include a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column unless otherwise stated. Stripping columns typically omit a reboiler at a bottom of the column and instead provide heating requirements and separation impetus from a fluidized inert media such as steam.

As used herein, the term “True Boiling Point” (TBP) means a test method for determining the boiling point of a material which corresponds to ASTM D-2892 for the production of a liquefied gas, distillate fractions, and residuum of standardized quality on which analytical data can be obtained, and the determination of yields of the above fractions by both mass and volume from which a graph of temperature versus mass % distilled is produced using fifteen theoretical plates in a column with a 5:1 reflux ratio.

As used herein, the term “initial boiling point” (IBP) means the temperature at which the sample begins to boil using ASTM D86.

As used herein, the term “T5” or “T95” means the temperature at which 5 volume percent or 95 volume percent, as the case may be, respectively, of the sample boils using ASTM D-86.

As used herein, the term “diesel cut point” is between about 343° C. (650° F.) and about 399° C. (750° F.) using the TBP distillation method.

As used herein, the term “diesel boiling range” means hydrocarbons boiling with an IBP in the range of between about 132° C. (270° F.) and about 210° C. (410° F.) and the diesel cut point using the TBP distillation method.

As used herein, the term “diesel conversion” means conversion of feed to material that boils at or below the diesel cut point of the diesel boiling range.

As used herein, the term “kerosene boiling range” means hydrocarbons boiling with an IBP in the range of between about 120° C. (248° F.) and about 150° C. (302° F.) and a kerosene cut point in the range of between about 132° C. (270° F.) and about 260° C. (500° F.) using the TBP distillation method.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator which latter may be operated at higher pressure.

As used herein, the term “predominant” or “predominate” means greater than 50%, suitably greater than 75% and preferably greater than 90%.

### DETAILED DESCRIPTION

The subject process and apparatus locates the hydroisomerization reactor downstream of the hydroprocessing unit. The hydroisomerization reactor may be moved downstream of a stripper into a lower pressure section of the flow scheme. Hydroisomerization reactions are favored at lower pressure, so operating the hydroisomerization outside of the high pressure hydroprocessing section is advantageous. All the product specifications such as diesel color, cetane, API, sulfur and nitrogen concentrations can still be met as an upstream hydrotreating or hydrocracking reactor is operated at much higher hydrogen partial pressure. Operating the hydroisomerization reactor at lower pressure lowers the capital and operational expense. The hydroprocessing reactors can be fully loaded with hydroprocessing catalyst without ceding volume to hydroisomerization catalyst that is instead loaded in a dedicated downstream reactor. Pour point reduction of 20-25 degrees Celsius can be achieved with a reasonable cycle length either matching or exceeding the upstream hydroprocessing reactor.

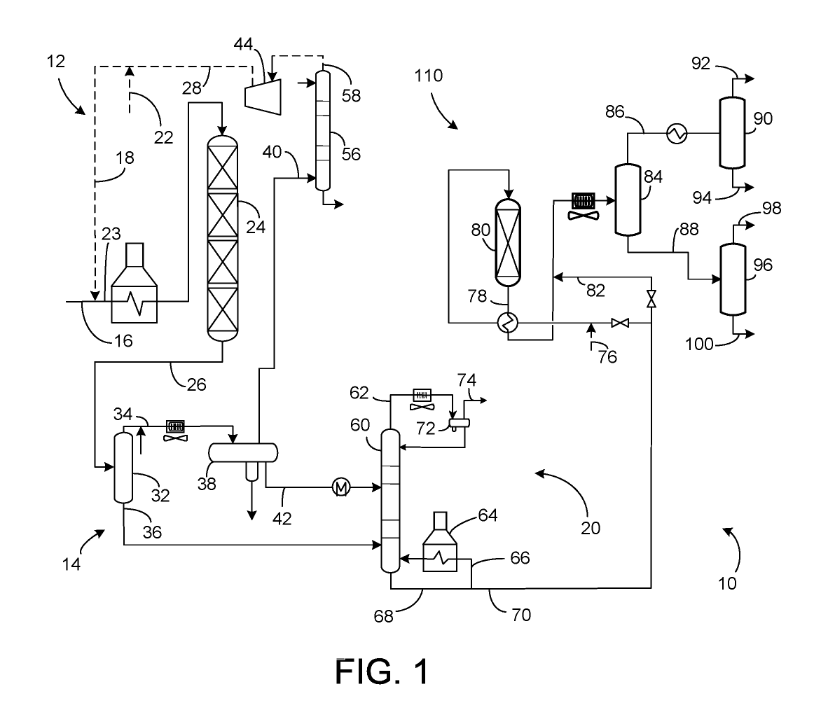

In FIG. 1, the hydroprocessing unit 10 for hydroprocessing hydrocarbons comprises a hydrotreating unit 12, a separation section 14, a product recovery section 20 and a hydroisomerization unit 110. A hydrocarbonaceous stream in hydrocarbon line 16 and a hydrogen stream in hydrogen line 18 are fed to the hydrotreating unit 12. Hydroprocessing effluent is separated in the separation section 14 and fractionated in the product recovery section 20.

A recycle hydrogen stream in recycle hydrogen line 28 may be supplemented by a make-up hydrogen stream from line 22 to provide the hydrogen stream in hydrogen line 18. The hydrogen stream may join the hydrocarbonaceous stream in feed line 16 to provide a hydrocarbon feed stream in feed line 23. The hydrocarbon feed stream in feed line 23 may be heated in a fired heater and fed to a hydroprocessing reactor which is a hydrotreating reactor 24. The hydrocarbon feed stream is hydroprocessed in a hydroprocessing reactor which is a hydrotreating reactor 24. Specifically, the hydrocarbon feed stream is hydrotreated in the hydrotreating reactor 24.

In one aspect, the process and apparatus described herein are particularly useful for hydrotreating a hydrocarbon feed stream comprising a feedstock boiling in the diesel range. Preferred feedstocks include straight run diesel from a crude column which may include materials boiling in the kerosene boiling range. Feedstock boiling in the kerosene range may also be suitable feed to the process. The feedstock can be termed a distillate feed stock.

Hydrotreating is a process wherein hydrogen is contacted with hydrocarbon in the presence of suitable catalysts which are primarily active for the removal of heteroatoms, such as sulfur, nitrogen and metals from the hydrocarbon feedstock. In hydrotreating, hydrocarbons with double and triple bonds may be saturated. Aromatics may also be saturated. Some hydrotreating processes are specifically designed to saturate aromatics. Consequently, the term “hydroprocessing” will include the term “hydrotreating” herein.

The hydrotreating reactor 24 may be a fixed bed reactor that comprises one or more vessels, single or multiple beds of catalyst in each vessel, and various combinations of hydrotreating catalyst in one or more vessels. It is contemplated that the hydrotreating reactor 24 be operated in a continuous liquid phase in which the volume of the liquid hydrocarbon feed is greater than the volume of the hydrogen gas. The hydrotreating reactor 24 may also be operated in a conventional continuous gas phase, a moving bed or a fluidized bed hydrotreating reactor. The hydrotreating reactor 24 may provide conversion per pass of about 10 to about 30 vol %.

The hydrotreating reactor 24 may comprise a guard bed of hydrotreating catalyst followed by one or more beds of higher quality hydrotreating catalyst. The guard bed filters particulates and picks up contaminants in the hydrocarbon feed stream such as metals like nickel, vanadium, silicon and arsenic which deactivate the catalyst. The guard bed may comprise material similar to the hydrotreating catalyst. Supplemental hydrogen may be added at an interstage location between catalyst beds in the hydrotreating reactor 24.

Suitable hydrotreating catalysts are any known conventional hydrotreating catalysts and include those which are comprised of at least one Group VIII metal, preferably iron, cobalt and nickel, more preferably cobalt and/or nickel and at least one Group VI metal, preferably molybdenum and tungsten, on a high surface area support material, preferably alumina. Other suitable hydrotreating catalysts include zeolitic catalysts, as well as noble metal catalysts where the noble metal is selected from palladium and platinum. It is within the scope of the present description that more than one type of hydrotreating catalyst be used in the same hydrotreating reactor 24. The Group VIII metal is typically present in an amount ranging from about 2 to about 20 wt %, preferably from about 4 to about 12 wt %. The Group VI metal will typically be present in an amount ranging from about 1 to about 25 wt %, preferably from about 2 to about 25 wt %.

Preferred hydrotreating reaction conditions include a temperature from about 290° C. (550° F.) to about 455° C. (850° F.), suitably 316° C. (600° F.) to about 427° C. (800° F.) and preferably 343° C. (650° F.) to about 399° C. (750° F.), a pressure from about 4.1 MPa (gauge) (600 psig) to about 11.0 MPa (gauge) (1600 psig), a liquid hourly space velocity of the fresh hydrocarbonaceous feedstock from about 0.1 hr⁻¹, suitably 0.5 hr⁻¹, to about 5 hr⁻¹, preferably from about 1.5 to about 4 hr⁻¹, and a hydrogen rate of about 84 Nm³/m³ (500 scf/bbl), to about 1,011 Nm³/m³ oil (6,000 scf/bbl), preferably about 168 Nm³/m³ oil (1,000 scf/bbl) to about 674 Nm³/m³ oil (4,000 scf/bbl), with a hydrotreating catalyst or a combination of hydrotreating catalysts.

The hydrotreating reactor 24 provides a hydroprocessing effluent stream that exits the hydrotreating reactor 24 in a hydroprocessing effluent line 26. The hydroprocessing effluent stream comprises material that will be separated in a separation section 14 comprising one or more separators into a liquid hydrotreated stream and a gaseous hydrotreated stream. The separation section 14 is in downstream communication with the hydrotreating reactor 24.

The hydroprocessing effluent stream in hydroprocessing effluent line 26 may in an aspect be heat exchanged with the hydrocarbon feed stream in line 16 to be cooled before entering a hot separator 32. The hot separator 32 separates the hydrotreating effluent to provide a hydrocarbonaceous hot gaseous stream in an overhead line 34 and a hydrocarbonaceous hot liquid stream in a bottoms line 36. The hot separator 32 may be in downstream communication with the hydrotreating reactor 24. The hot separator 32 operates at about 177° C. (350° F.) to about 371° C. (700° F.) and preferably operates at about 232° C. (450° F.) to about 315° C. (600° F.). The hot separator 32 may be operated at a slightly lower pressure than the hydrotreating reactor 24 accounting for pressure drop of intervening equipment. The hot separator may be operated at pressures around that of the hydrotreating reactor 24 less frictional losses. The liquid hydrocarbonaceous hot liquid stream 36 may have a temperature of the operating temperature of the hot separator 32.

The hot gaseous stream in the overhead line 34 may be cooled before entering a cold separator 38. As a consequence of the reactions taking place in the hydrotreating reactor 24 wherein nitrogen, chlorine and sulfur are removed from the feed, ammonia and hydrogen sulfide are formed. At a characteristic temperature, ammonia and hydrogen sulfide will combine to form ammonium bisulfide and ammonia and chlorine will combine to form ammonium chloride. Each compound has a characteristic sublimation temperature that may allow the compound to coat equipment, particularly heat exchange equipment, impairing its performance. To prevent such deposition of ammonium bisulfide or ammonium chloride salts in the line 34 transporting the hot gaseous stream, a suitable amount of wash water may be introduced into line 34 upstream of a cooler at a point in line 34 where the temperature is above the characteristic sublimation temperature of either compound.

The hot gaseous stream may be separated in the cold separator 38 to provide a cold gaseous stream comprising a hydrogen-rich gas stream in an overhead line 40 and a cold liquid stream in a cold bottoms line 42. The cold separator 38 serves to separate hydrogen from hydrocarbon in the hydrotreating effluent for recycle to the hydrotreating reactor 24 in the cold overhead line 40. The cold separator 38, therefore, is in downstream communication with the overhead line 34 of the hot separator 32 and the hydrotreating reactor 24. The cold separator 38 may be operated at about 100° F. (38° C.) to about 150° F. (66° C.), suitably about 115° F. (46° C.) to about 145° F. (63° C.), and just below the pressure of the hydrotreating reactor 24 and the hot separator 32 accounting for pressure drop of intervening equipment to keep hydrogen and light gases in the overhead and normally liquid hydrocarbons in the bottoms. The cold separator 38 may also have a boot for collecting an aqueous phase. The cold liquid stream may have a temperature of the operating temperature of the cold separator 38.

The hydrocarbonaceous hot liquid stream in the hot bottoms line 36 may be let down in pressure and stripped as hot hydrotreating effluent stream in a stripper column 60. In an aspect, the hot liquid stream in the hot bottoms line 36 may be let down in pressure and flashed in a hot flash drum (not shown) to reduce the pressure of the hot liquid stream in line 36.

In an aspect, the cold liquid stream in the cold bottoms line 42 is stripped as a cold hydrotreating effluent stream in the stripper column 60. In a further aspect, the cold liquid stream may be let down in pressure and flashed in a cold flash drum (not shown) to reduce the pressure of the cold liquid stream in the bottoms line 42. A cold aqueous stream may be removed from a boot in the cold separator 38.

The cold gaseous stream in the overhead line 40 is rich in hydrogen. Thus, hydrogen can be recovered from the cold gaseous stream. The cold gaseous stream in overhead line 40 may be passed through a trayed or packed recycle scrubbing column 56 where it is scrubbed by means of a scrubbing extraction liquid such as an aqueous amine solution to remove acid gases including hydrogen sulfide by extracting them into the aqueous solution. In the recycle scrubber column 56, the cold gaseous stream enters the recycle scrubber column 56 at an inlet near a bottom and flows upwardly, while a lean amine stream in a solvent line enters the stripper scrubber column at an inlet near a top and flows downwardly. Preferred lean amines include alkanolamines DEA, MEA, and MDEA. Other amines can be used in place of or in addition to the preferred amines. The spent scrubbing liquid from the bottoms may be regenerated and recycled back to the recycle scrubbing column 56. The scrubbed hydrogen-rich stream emerges from the scrubber via an overhead line 58 and may be compressed in a recycle compressor to provide a recycle hydrogen stream in recycle line 28. The recycle hydrogen stream in the recycle line 28 may be supplemented with a make-up hydrogen stream in make-up line 22 to provide the hydrogen stream in hydrogen line 18. A portion of the material in line 28 may be routed to the intermediate catalyst bed outlets in the hydrotreating reactor 24 to control the inlet temperature of the subsequent catalyst bed (not shown).

The product recovery section 20 may include a stripping column 60. The stripping column 60 may be in downstream communication with a bottoms line in the separation section 14. For example, the stripping column 60 may be in downstream communication with the hydrotreating reactor 24, the hot bottoms line 36 and/or the cold bottoms line 42. In an aspect, the stripping column 60 may comprise two stripping columns. The stripping column 60 may be in direct, downstream communication with the cold bottoms line 42 for stripping the entire cold hydrotreating liquid stream. The stripping column 60 may be in direct, downstream communication with the hot bottoms line 36 for stripping an entire hot hydrotreating liquid stream which is hotter than the cold hydrotreating liquid stream. The hot hydrotreating liquid stream is hotter than the cold hydrotreating liquid stream, by at least 25° C. and preferably at least 50° C.

The cold hydrotreating liquid stream may be heated and fed to the stripping column 60 at a location that may be in the top half of the stripping column 60. The hot hydrotreating effluent stream may be heated and fed to the stripping column 60 at a location that may be in the bottom half of the stripping column 60. The cold hydrotreating effluent stream and the hot hydrotreating effluent stream which each comprise at least a portion of the hydrotreating effluent stream may be stripped of light gases in the stripping column 60 which has a reboiler 64. The reboiler 64 receives a portion of the bottom stream 66 in reboil line 68, reboils it and sends it back to the bottom of the stripping column 60. Stripping media which is an inert gas such as steam from a stripping media line is preferably not used to avoid adding water to the stripping column and he bottoms product in line 68. The reboiled stream in line 66 may comprise no more than about 1 wt % water. The reboiler 64 may be a fired heater or a heat exchanger. The stripping column 60 provides an overhead off gas stream of naphtha, hydrogen, hydrogen sulfide, steam and other gases in a stripper overhead line 62. The stripping column 60 strips light gases from the hot liquid stream and/or the cold liquid stream to provide a stripper off gas stream and a stripped hydrotreated stream in a stripped bottoms line 70.

At least a portion of the stripper overhead off gas stream may be condensed and separated in a receiver 72. A stripper net overhead line 74 from the receiver 72 carries a net stripper off gas stream. The stripper may be run at total reflux, so all condensed material may be refluxed to the column. Alternatively, unstabilized liquid naphtha from the bottoms of the receiver 72 may be split between a reflux portion refluxed to the top of the stripping column 60 and a stripper overhead liquid stream which may be recovered, but the stripper overhead liquid stream is not shown. A sour water stream (not shown) may be collected from a boot of the overhead receiver 72.

The stripping column 60 may be operated with a bottoms temperature between about 160° C. (320° F.) and about 360° C. (680° F.), and an overhead pressure of about 0.35 MPa (gauge) (50 psig), preferably about 0.70 MPa (gauge) (100 psig), to about 2.0 MPa (gauge) (300 psig). The temperature in the overhead receiver 72 ranges from about 38° C. (100° F.) to about 66° C. (150° F.) and the pressure is essentially the same as in the overhead of the stripping column 60.

Typically, the stripped hydroprocessed stream in a stripped bottoms line 70 comprises predominantly diesel range boiling material because the feed to the hydrotreating unit 24 predominantly boils in the diesel range. However, when the feed stream to the hydrotreating reactor 24 is a predominantly a kerosene stream, a stripped hydrotreated stream in the stripped bottoms line 70 comprises predominantly kerosene range boiling material. The stripped hydrotreated stream may have an end point between about 343° C. (650° F.) and about 399° C. (750° F.) and may have an IBP in the range of between about 132° C. (270° F.) and about 210° C. (410° F.).

During the colder months, the cold flow properties of the distillate in the stripped hydroprocessed stream in the stripped bottoms line 70 may need improvement to meet winter specifications. Consequently, the stripped hydroprocessed distillate stream is treated in the hydroisomerization unit 110. A valve on the stripped bottoms line 70 is opened to allow passage to the hydroisomerization unit 110, and the stripped hydroprocessed distillate stream in the stripped bottoms line 70 is supplemented with a hydroisomerization hydrogen stream taken from a make-up gas stream in a hydroisomerization hydrogen line 76. The hydroisomerization hydrogen stream may be heated in a steam heater (not shown) to adjust the temperature of the stripped hydroprocessed distillate stream. The stripped hydroprocessed distillate stream mixed with the hydroisomerization hydrogen stream may be heat exchanged with hydroisomerized effluent in a hydroisomerized effluent line 78 and fed to a hydroisomerization reactor 80. The hydroisomerization hydrogen stream is not on the recycle gas loop that includes the recycle gas compressor 44, so the pressure in the hydroisomerization rector 80 may be reduced relative to the upstream hydroprocessing reactor.

The stripped hydroprocessed stream comprising distillate is hydroisomerized over a hydroisomerization catalyst bed in the presence of the hydroisomerization hydrogen stream to provide a hydroisomerized stream. Only a single hydroisomerization catalyst bed is shown in hydroisomerization reactor 80, but additional hydroisomerization catalyst beds may be located in the hydroisomerization reactor 80.

The hydroisomerization catalyst can comprise an unbound 10-member ring pore, one-dimensional zeolite in combination with a low surface area metal oxide refractory binder, both of which are selected to obtain a high ratio of micropore surface area to total surface area. Alternatively, the zeolite has a low silica to alumina ratio. Suitable catalysts include 10-member ring pore zeolites, such as EU-1, ZSM-35 (or ferrierite), ZSM-11, ZSM-57, NU-87, SAPO-11, and ZSM-22. Preferred materials are EU-2, EU-11, ZBM-30, ZSM-48, or ZSM-23. ZSM-48 is most preferred. Note that a zeolite having the ZSM-23 structure with a silica to alumina ratio of from about 20:1 to about 40:1 can sometimes be referred to as SSZ-32. Other molecular sieves that are isostructural with the above materials include Theta-1, NU-10, EU-13, KZ-1, and NU-23.

The hydroisomerization catalyst can further include a metal hydrogenation function, such as a Group VI or Group VIII metal, and suitably a Group VIII noble metal. The metal hydrogenation component is typically a Group VI and/or a Group VIII metal. The metal hydrogenation component may be a Group VIII noble metal. Preferably, the metal hydrogenation component is a combination of a non-noble Group VIII metal with a Group VI metal. Suitable combinations can include nickel, cobalt, or iron with molybdenum or tungsten, preferably nickel with molybdenum or tungsten.

The metal hydrogenation component may be added to the catalyst in any convenient manner. One technique for adding the metal hydrogenation component is by incipient wetness. For example, after combining a zeolite and a binder, the combined zeolite and binder can be extruded into catalyst particles. These catalyst particles can then be exposed to a solution containing a suitable metal precursor. Alternatively, metal can be added to the catalyst by ion exchange, where a metal precursor is added to a mixture of zeolite (or zeolite and binder) prior to extrusion.

The amount of metal in the catalyst can be at least about 0.1 wt % to about 10 wt % based on catalyst. Preferably, the hydroisomerization catalysts has a low ratio of silica to alumina. In various embodiments, the ratio of silica to alumina can be from 30:1 to 200:1, 60:1 to 110:1, or 70:1 to 100:1. The hydroisomerization catalysts may also include an optional binder having a low surface area such as 100 m²/g or less, or 80 m²/g or less, or 70 m²/g or less. A zeolite can be combined with binder by starting with powders of both the zeolite and binder, combining and mulling the powders with added water to form a mixture, and then extruding the mixture to produce a bound catalyst of a desired size. Extrusion aids can also be used to modify the extrusion flow properties of the zeolite and binder mixture. The amount of framework alumina in the catalyst may range from 0.1 to 3.33 wt %, or 0.1 to 2.7 wt %, or 0.2 to 2 wt %, or 0.3 to 1 wt %.

Process conditions in the hydroisomerization reactor may include a temperature of from 200 to 450° C., suitably 250 to 400° C., and preferably 250 to 350° C., a pressure of about 1.7 MPa (250 psig) to about 3.1 MPa (450 psig), preferably about 2.1 MPa (300 psig) to about 2.8 MPa (400 psig), a hydrogen partial pressure of from about 1.5 MPa (218 psig) to 3 MPa (435 psig), preferably about 1.7 MPa (250 psig) to about 2.6 MPa (380 psig), a liquid hourly space velocity of from about 1 to about 4 v/v/hr, preferably about 2 to about 3 v/v/hr, and a hydrogen circulation rate of from 35.6 Nm³/m³ (200 scf/B), to 200 Nm³/m³ (1150 scf/B), preferably 80 Nm³/m³ (450 scf/B) to 150 Nm³/m³ (850 scf/B). The hydroisomerization process is conducted at a lower pressure than the hydroprocessing process because hydroisomerization reactions are favored at lower pressure. In an aspect, the hydroprocessing reaction is run at a pressure that is at least about 1.4 MPa (200 psi) greater than the hydroisomerization reaction. In a further aspect, the hydroprocessing reaction is run at a pressure that is at least about 2.1 MPa (300 psi) greater than the hydroisomerization reaction. Because the hydroisomerization reactor runs at lower pressure, it can be made of lower grade steel such as 1.25 Cr/0.5 Mo.

In warmer months, when cold flow specifications are not as stringent, the stripped hydroprocessed stream in line 70 can bypass the hydroisomerization reactor 80 in bypass line 82 with the valve thereon opened and the valve on the stripped bottoms line 70 closed. During bypassing, the isomerization reactor 80 can be shut down with the flow of the stripped hydroprocess stream in the stripped bottoms line 70 to the hydroisomerization reactor 80 terminated. The bypass line 82 may be in downstream communication with the stripper column 60 but be out of upstream communication with said hydroisomerization reactor 80 for bypassing the stripped hydroprocessed liquid stream around the hydroisomerization reactor.

These valves can also be adjusted to allow more or less stripped hydroprocessed distillate in the stripped bottoms line 70 to the hydroisomerization reactor 80 or less depending upon the circumstances.

The hydroisomerized effluent in hydroisomerized effluent line 78 may be heat exchanged with the stripped hydroprocessed liquid stream in the stripped bottoms line 70 to cool it, be further cooled and be fed to a hydroisomerization hot separator 84 to provide a hydroisomerized vapor stream in the hot hydroisomerization overhead line and a hydroisomerized liquid stream in a hot hydroisomerization bottoms line 88. The hot hydroisomerization separator operates at about the same pressure as the hydroisomerization reactor and between about 100 and about 150° C. The hydroisomerized vapor stream is further cooled and sent to a hydroisomerization cold separator 90, which typically operates at ambient temperature, preferably between about 25 and about 50° C., preferably between about 30 and about 40° C.

The hydroisomerization cold separator 90 separates the hydroisomerized vapor stream in the hot hydroisomerization overhead line 86 into a cold hydroisomerization vapor stream containing unconsumed hydrogen gas in cold diesel overhead line 92 and a hydroisomerized naphtha stream in a bottoms line 94 which can be forwarded to fractionation such as to a debutanizer column to produce high octane naphtha or can be routed to the upstream net stripper off gas stream in the stripper net overhead line 74. The cold hydroisomerization vapor stream may be forwarded to a make-up gas compressor to reuse the unconsumed hydrogen.

The hydroisomerized liquid stream in the hot hydroisomerization bottoms line 88 may be separated in a hydroisomerization hot flash drum 96 to provide a fuel gas stream in the hydroisomerization hot flash overhead line 98 and a hydroisomerized diesel product stream in diesel hot flash bottoms line 100 which can be forwarded to a diesel pool or for diesel rundown via rundown cooler (not shown). The hydroisomerized diesel in the hot flash bottoms line 100 may have a cloud point reduction of 20 to 25° C. with a reasonable cycle length of 2 to 5 years.

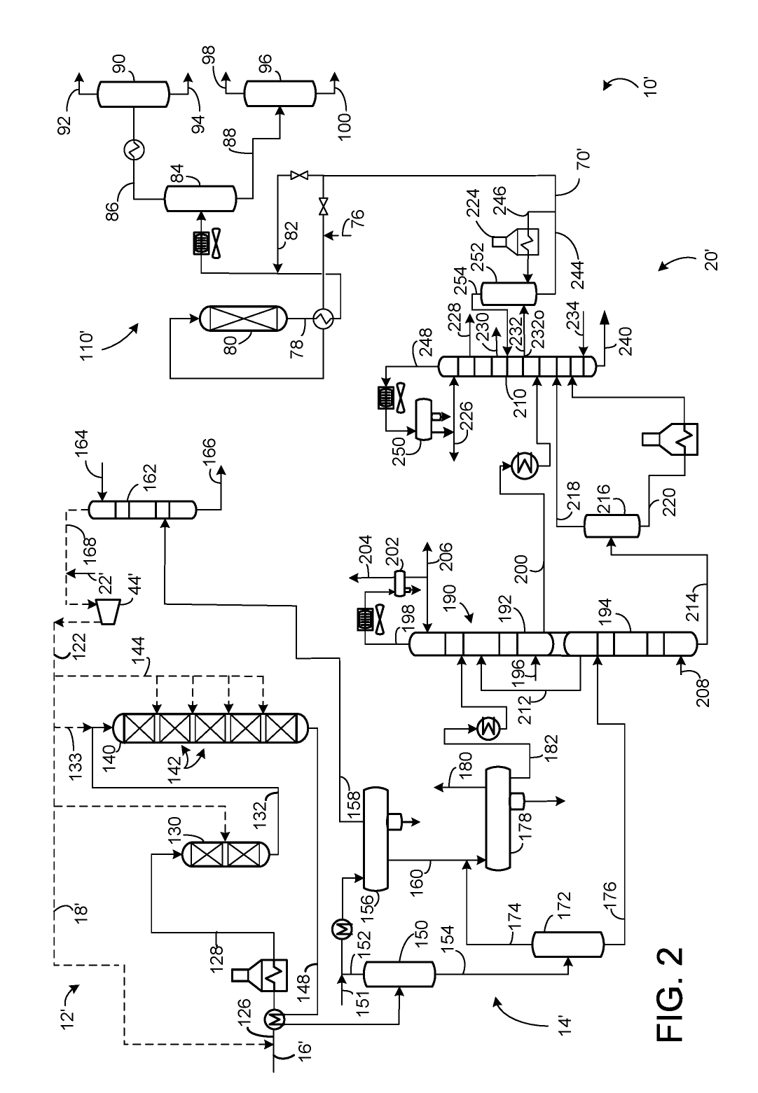

In FIG. 2, the hydroprocessing 10′ unit for hydroprocessing hydrocarbons comprises a hydrocracking unit 12′, a separator section 14′, a product recovery unit 20′, and a hydroisomerization unit 110′. A hydrocarbonaceous stream in hydrocarbon line 16′ and a hydrogen stream in hydrogen line 18′ are fed to the hydrocracking unit 12′. Hydroprocessing effluent is separated in the separation section 14′ and fractionated in the product recovery section 20′. The hydroprocessing reactor in FIG. 2 may be a hydrocracking reactor 140.

In one aspect, the process and apparatus described herein are particularly useful for hydrocracking a hydrocarbon feed stream comprising a hydrocarbonaceous feedstock.

Illustrative hydrocarbonaceous feed stocks include hydrocarbon streams having initial boiling points (IBP) above about 288° C. (550° F.), such as atmospheric gas oils, vacuum gas oil (VGO) having T5 and T95 between about 315° C. (600° F.) and about 600° C. (1100° F.), deasphalted oil, coker distillates, straight run distillates, pyrolysis-derived oils, high boiling synthetic oils, cycle oils, clarified slurry oils, deasphalted oil, shale oil, hydrocracked feeds, catalytic cracker distillates, atmospheric residue having an IBP at or above about 343° C. (650° F.) and vacuum residue having an IBP above about 510° C. (950° F.).

The hydrogen stream in the hydrogen line 18′ may split off from a hydroprocessing hydrogen line 122. The hydrotreating hydrogen stream may join the hydrocarbonaceous stream in feed line 18′ to provide a hydrocarbon feed stream in a hydrocarbon feed line 126. The hydrocarbon feed stream in the hydrocarbon feed line 126 may be heated by heat exchange with a hydrocracked stream in line 148 and in a fired heater. The heated hydrocarbon feed stream in line 128 may be fed to an optional hydrotreating reactor 130. The hydrotreating reactor 130 may be operated under the same or similar conditions and catalyst as described with respect to FIG. 1 for the hydrotreating reactor 24.

The hydrocarbon feed stream in the hydrocarbon feed line 128 may be hydrotreated over the hydrotreating catalyst in the hydrotreating reactor 130 to provide a hydrotreated hydrocarbon feed stream that exits the hydrotreating reactor 130 in a hydrotreating effluent line 132 which can be taken as a hydrocracking feed stream. The hydrogen gas laden with ammonia and hydrogen sulfide may be removed from the hydrocracking feed stream in a separator, but the hydrocracking feed stream is typically fed directly to the hydrocracking reactor 140 without separation. The hydrocracking feed stream may be mixed with a hydrocracking hydrogen stream in a hydrocracking hydrogen line 133 from the hydroprocessing hydrogen line 122 and be fed through an inlet to the hydrocracking reactor 140 to be hydrocracked.

Hydrocracking is a process in which hydrocarbons crack in the presence of hydrogen to lower molecular weight hydrocarbons. The hydrocracking reactor 140 may be a fixed bed reactor that comprises one or more vessels, single or multiple catalyst beds 142 in each vessel, and various combinations of hydrotreating catalyst and/or hydrocracking catalyst in one or more vessels. It is contemplated that the hydrocracking reactor 140 be operated in a continuous liquid phase in which the volume of the liquid hydrocarbon feed is greater than the volume of the hydrogen gas. The hydrocracking reactor 140 may also be operated in a conventional continuous gas phase, a moving bed or a fluidized bed hydroprocessing reactor. The term “hydroprocessing” will include the term “hydrocracking” herein.

The hydrocracking reactor 140 comprises a plurality of hydrocracking catalyst beds 142. If the hydrocracking unit 12′ does not include a hydrotreating reactor 130, the catalyst beds 142 in the hydrocracking reactor 140 may include a hydrotreating catalyst for the purpose of saturating, demetallizing, desulfurizing or denitrogenating the hydrocarbon feed stream before it is hydrocracked with the hydrocracking catalyst in subsequent vessels or catalyst beds 142 in the hydrocracking reactor 140.

The hydrotreated hydrocarbon feed stream is hydroprocessed over a hydroprocessing catalyst in a hydroprocessing reactor in the presence of a hydrocracking hydrogen stream from a hydrocracking hydrogen line 133 to provide a hydroprocessing effluent stream. Specifically, the hydrotreated hydrocarbon feed stream is hydrocracked over a hydrocracking catalyst in the hydrocracking reactor 140 in the presence of the hydrocracking hydrogen stream from a hydrocracking hydrogen line 133 to provide a hydrocracking effluent stream. Hydrogen manifold 144 may deliver supplemental hydrogen streams to one, some or each of the catalyst beds 142. In an aspect, the supplemental hydrogen is added to each of the hydrocracking catalyst beds 142 at an interstage location between adjacent beds, so supplemental hydrogen is mixed with hydroprocessed effluent exiting from the upstream catalyst bed 142 before entering the downstream catalyst bed 142.

The hydrocracking reactor may provide a total conversion of at least about 20 vol % and typically greater than about 60 vol % of the hydrocracking feed stream in the hydrotreating effluent line 132 to products boiling below the diesel cut point. The hydrocracking reactor 40 may operate at partial conversion of more than about 30 vol % or full conversion of at least about 90 vol % of the feed based on total conversion. The hydrocracking reactor 40 may be operated at mild hydrocracking conditions which will provide about 20 to about 60 vol %, preferably about 20 to about 50 vol %, total conversion of the hydrocarbon feed stream to product boiling below the diesel cut point.

The hydrocracking catalyst may utilize amorphous silica-alumina bases or low-level zeolite bases combined with one or more Group VIII or Group VIB metal hydrogenating components if mild hydrocracking is desired to produce a balance of middle distillate and gasoline. In another aspect, when middle distillate is significantly preferred in the converted product over gasoline production, partial or full hydrocracking may be performed in the hydrocracking reactor 140 with a catalyst which comprises, in general, any crystalline zeolite cracking base upon which is deposited a Group VIII metal hydrogenating component. Additional hydrogenating components may be selected from Group VIB for incorporation with the zeolite base.

The zeolite cracking bases are sometimes referred to in the art as molecular sieves and are usually composed of silica, alumina and one or more exchangeable cations such as sodium, magnesium, calcium, rare earth metals, etc. They are further characterized by crystal pores of relatively uniform diameter between about 4 and about 14 Angstroms (10⁻¹⁰ meters). It is preferred to employ zeolites having a relatively high silica/alumina mole ratio between about 3 and about 12. Suitable zeolites found in nature include, for example, mordenite, stilbite, heulandite, ferrierite, dachiardite, chabazite, erionite and faujasite. Suitable synthetic zeolites include, for example, the B, X, Y and L crystal types, e.g., synthetic faujasite and mordenite. The preferred zeolites are those having crystal pore diameters between about 8 and 12 Angstroms (10⁻¹⁰ meters), wherein the silica/alumina mole ratio is about 4 to 6. One example of a zeolite falling in the preferred group is synthetic Y molecular sieve.

The natural occurring zeolites are normally found in a sodium form, an alkaline earth metal form, or mixed forms. The synthetic zeolites are nearly always prepared in the sodium form. In any case, for use as a cracking base it is preferred that most or all of the original zeolitic monovalent metals be ion-exchanged with a polyvalent metal and/or with an ammonium salt followed by heating to decompose the ammonium ions associated with the zeolite, leaving in their place hydrogen ions and/or exchange sites which have actually been decationized by further removal of water. Hydrogen or “decationized” Y zeolites of this nature are more particularly described in U.S. Pat. No. 3,100,006.

Mixed polyvalent metal-hydrogen zeolites may be prepared by ion-exchanging with an ammonium salt, then partially back exchanging with a polyvalent metal salt and then calcining. In some cases, as in the case of synthetic mordenite, the hydrogen forms can be prepared by direct acid treatment of the alkali metal zeolites. In one aspect, the preferred cracking bases are those which are at least about 10 wt %, and preferably at least about 20 wt %, metal-cation-deficient, based on the initial ion-exchange capacity. In another aspect, a desirable and stable class of zeolites is one wherein at least about 20 wt % of the ion exchange capacity is satisfied by hydrogen ions.

The active metals employed in the preferred hydrocracking catalysts of the present invention as hydrogenation components are those of Group VIII, i.e., iron, cobalt, nickel, ruthenium, rhodium, palladium, osmium, iridium and platinum. In addition to these metals, other promoters may also be employed in conjunction therewith, including the metals of Group VIB, e.g., molybdenum and tungsten. The amount of hydrogenating metal in the catalyst can vary within wide ranges. Broadly speaking, any amount between about 0.05 wt % and about 30 wt % may be used. In the case of the noble metals, it is normally preferred to use about 0.05 to about 2 wt % noble metal.

The method for incorporating the hydrogenation metal is to contact the base material with an aqueous solution of a suitable compound of the desired metal wherein the metal is present in a cationic form. Following addition of the selected hydrogenation metal or metals, the resulting catalyst powder is then filtered, dried, pelleted with added lubricants, binders or the like if desired, and calcined in air at temperatures of, e.g., about 371° C. (700° F.) to about 648° C. (1200° F.) in order to activate the catalyst and decompose ammonium ions. Alternatively, the base component may be pelleted, followed by the addition of the hydrogenation component and activation by calcining.

The foregoing catalysts may be employed in undiluted form, or the powdered catalyst may be mixed and copelleted with other relatively less active catalysts, diluents or binders such as alumina, silica gel, silica-alumina cogels, activated clays and the like in proportions ranging between about 5 and about 90 wt %. These diluents may be employed as such or they may contain a minor proportion of an added hydrogenating metal such as a Group VIB and/or Group VIII metal. Additional metal promoted hydrocracking catalysts may also be utilized in the process of the present invention which comprises, for example, aluminophosphate molecular sieves, crystalline chromosilicates and other crystalline silicates. Crystalline chromosilicates are more fully described in U.S. Pat. No. 4,363,718.

By one approach, the hydrocracking conditions may include a temperature from about 290° C. (550° F.) to about 468° C. (875° F.), preferably 343° C. (650° F.) to about 445° C. (833° F.), a pressure from about 4.8 MPa (gauge) (700 psig) to about 20.7 MPa (gauge) (3000 psig), a liquid hourly space velocity (LHSV) from about 0.4 to less than about 2.5 hr⁻¹ and a hydrogen rate of about 421 Nm³/m³ (2,500 scf/bbl) to about 2,527 Nm³/m³ oil (15,000 scf/bbl). If mild hydrocracking is desired, conditions may include a temperature from about 315° C. (600° F.) to about 441° C. (825° F.), a pressure from about 5.5 MPa (gauge) (800 psig) to about 13.8 MPa (gauge) (2000 psig) or more typically about 6.9 MPa (gauge) (1000 psig) to about 11.0 MPa (gauge) (1600 psig), a liquid hourly space velocity (LHSV) from about 0.5 to about 2 hr⁻¹ and preferably about 0.7 to about 1.5 hr⁻¹ and a hydrogen rate of about 421 Nm³/m³ oil (2,500 scf/bbl) to about 1,685 Nm³/m³ oil (10,000 scf/bbl).

The hydroprocessed effluent stream may exit the hydrocracking reactor 140 in the hydrocracking effluent line 148 and be separated in the separation section 14′ in downstream communication with the hydrocracking reactor 140. The separation section 14′ comprises one or more separators in downstream communication with the hydrocracking reactor 140. The hydrocracked stream in the hydrocracked line 148 may in an aspect be heat exchanged with the hydrocarbon feed stream in the feed line 126 and be delivered to a hot separator 150.

The hot separator 150 separates the hydrocracking effluent stream to provide a hydrocarbonaceous, hot gaseous stream in a hot overhead line 152 and a hydrocarbonaceous, hot liquid stream in a hot bottoms line 154. The hot separator 150 may be in downstream communication with the hydrocracking reactor 140. The hot separator 150 operates at about 177° C. (350° F.) to about 371° C. (700° F.) and preferably operates at about 232° C. (450° F.) to about 315° C. (600° F.). The hot separator 150 may be operated at a slightly lower pressure than the hydrocracking reactor 140 accounting for pressure drop through intervening equipment. The hot separator 150 may be operated at pressures between about 4.8 MPa (gauge) (700 psig) and about 20.4 MPa (gauge) (2959 psig). The hydrocarbonaceous, hot gaseous separated stream in the hot overhead line 152 may have a temperature of the operating temperature of the hot separator 150.

The hot gaseous stream in the hot overhead line 152 may be cooled before entering a cold separator 156. As a consequence of the reactions taking place in the hydrocracking reactor 140 wherein nitrogen, chlorine and sulfur are removed from the feed, ammonia, hydrogen chloride and hydrogen sulfide are formed. At a characteristic sublimation temperature, ammonia and hydrogen sulfide will combine to form ammonium bisulfide and ammonia, and hydrogen chloride will combine to form ammonium chloride. Each compound has a characteristic sublimation temperature that may allow the compound to coat equipment, particularly heat exchange equipment, impairing its performance. To prevent such deposition of ammonium bisulfide or ammonium chloride salts in the hot overhead line 152 transporting the hot gaseous stream, a suitable amount of wash water may be introduced into the hot overhead line 152 upstream of a cooler by water line 151 at a point in the hot overhead line where the temperature is above the characteristic sublimation temperature of either compound.

The hot gaseous stream may be separated in the cold separator 156 to provide a cold gaseous stream comprising a hydrogen-rich gas stream in a cold overhead line 158 and a cold liquid stream in a cold bottoms line 160. The cold separator 156 serves to separate hydrogen rich gas from hydrocarbon liquid in the hydrocracked stream for recycle to the hydrocracking unit 12′ in the cold overhead line 158. The cold separator 156, therefore, is in downstream communication with the hot overhead line 152 of the hot separator 150 and the hydrocracking reactor 140. The cold separator 156 may be operated at about 100° F. (38° C.) to about 150° F. (66° C.), suitably about 115° F. (46° C.) to about 145° F. (63° C.), and just below the pressure of the hydrocracking reactor 140 and the hot separator 150 accounting for pressure drop through intervening equipment to keep hydrogen and light gases in the overhead and normally liquid hydrocarbons in the bottoms. The cold separator 156 may be operated at pressures between about 4.8 MPa (gauge) (700 psig) and about 20 MPa (gauge) (2,901 psig). The cold separator 156 may also have a boot for collecting an aqueous phase. The cold liquid stream in the cold bottoms line 160 may have a temperature of the operating temperature of the cold separator 156.

The cold gaseous stream in the cold overhead line 158 is rich in hydrogen. Thus, hydrogen can be recovered from the cold gaseous stream. The cold gaseous stream in the cold overhead line 158 may be passed through a trayed or packed recycle scrubbing column 162 where it is scrubbed by means of a scrubbing extraction liquid such as an aqueous solution fed by line 164 to remove acid gases including hydrogen sulfide by extracting them into the aqueous solution. Preferred aqueous solutions include lean amines such as alkanolamines DEA, MEA, and MDEA. Other amines can be used in place of or in addition to the preferred amines. The lean amine contacts the cold gaseous stream and absorbs acid gas contaminants such as hydrogen sulfide. The resultant “sweetened” cold gaseous stream is taken out from an overhead outlet of the recycle scrubber column 162 in a recycle scrubber overhead line 168, and a rich amine is taken out from the bottoms at a bottom outlet of the recycle scrubber column in a recycle scrubber bottoms line 166. The spent scrubbing liquid from the bottoms may be regenerated and recycled back to the recycle scrubbing column 162 in line 164. The scrubbed hydrogen-rich stream emerges from the scrubber via the recycle scrubber overhead line 168 and may be compressed in a recycle compressor 44′. The scrubbed hydrogen-rich stream in the scrubber overhead line 168 may be supplemented with make-up hydrogen stream in the make-up line 22′ upstream or downstream of the compressor 44′. The compressed hydrogen stream supplies hydrogen to the hydrogen stream in the hydrogen line 22′. The recycle scrubbing column 162 may be operated with a gas inlet temperature between about 38° C. (100° F.) and about 66° C. (150° F.) and an overhead pressure of about 3 MPa (gauge) (435 psig) to about 20 MPa (gauge) (2900 psig).

The hydrocarbonaceous hot liquid stream in the hot bottoms line 154 may be directly stripped. In an aspect, the hot liquid stream in the hot bottoms line 154 may be let down in pressure and flashed in a hot flash drum 172 to provide a flash hot gaseous stream of light ends in a flash hot overhead line 174 and a flash hot liquid stream in a flash hot bottoms line 176. The hot flash drum 172 may be in direct, downstream communication with the hot bottoms line 154 and in downstream communication with the hydrocracking reactor 140. In an aspect, light gases such as hydrogen sulfide may be stripped from the flash hot liquid stream in the flash hot bottoms line 176. Accordingly, a stripping column 190 may be in direct, downstream communication with the hot flash drum 180 and the hot flash bottoms line 176.

The hot flash drum 172 may be operated at the same temperature as the hot separator 150 but at a lower pressure of between about 1.4 MPa (gauge) (200 psig) and about 6.9 MPa (gauge) (1000 psig), suitably no more than about 3.8 MPa (gauge) (550 psig). The flash hot liquid stream in the flash hot bottoms line 176 may be further separated in the separation section 14′. The flash hot liquid stream in the flash hot bottoms line 176 may have a temperature of the operating temperature of the hot flash drum 172.

In an aspect, the cold liquid stream in the cold bottoms line 160 may be directly stripped. In a further aspect, the cold liquid stream may be let down in pressure and flashed in a cold flash drum 178 to separate the cold liquid stream in the cold bottoms line 160. The cold flash drum 178 may be in direct, downstream communication with the cold bottoms line 160 of the cold separator 156 and in downstream communication with the hydrocracking reactor 140.

In a further aspect, the flash hot gaseous stream in the flash hot overhead line 174 may be fractionated in the recovery unit 20′. In a further aspect, the flash hot gaseous stream may be cooled and also separated in the cold flash drum 178. The cold flash drum 178 may separate the cold liquid stream in line 160 and/or the flash hot gaseous stream in the flash hot overhead line 174 to provide a flash cold gaseous stream in a flash cold overhead line 180 and a flash cold liquid stream in a cold flash bottoms line 182. In an aspect, light gases such as hydrogen sulfide may be stripped from the flash cold liquid stream in the flash cold bottoms line 182. Accordingly, a stripping column 190 may be in downstream communication with the cold flash drum 178 and the cold flash bottoms line 182.

The cold flash drum 178 may be in downstream communication with the cold bottoms line 160 of the cold separator 156, the hot flash overhead line 174 of the hot flash drum 172 and the hydrocracking reactor 140. The flash cold liquid stream in the cold bottoms line 160 and the flash hot gaseous stream in the hot flash overhead line 174 may enter into the cold flash drum 178 either together or separately. In an aspect, the hot flash overhead line 174 joins the cold bottoms line 160 and feeds the flash hot gaseous stream and the cold liquid stream together to the cold flash drum 178. The cold flash drum 178 may be operated at the same temperature as the cold separator 156 but typically at a lower pressure of between about 1.4 MPa (gauge) (200 psig) and about 6.9 MPa (gauge) (1000 psig) and preferably between about 3.0 MPa (gauge) (435 psig) and about 3.8 MPa (gauge) (550 psig). A flashed aqueous stream may be removed from a boot in the cold flash drum 178. The flash cold liquid stream in the flash cold bottoms line 182 may have the same temperature as the operating temperature of the cold flash drum 178. The flash cold gaseous stream in the flash cold overhead line 180 contains substantial hydrogen that may be recovered.

The fractionation unit 20′ may include the stripping column 190 and a fractionation column 210. The stripping column 190 may be in downstream communication with a separator 150, 172, 156, 178 or a bottoms line in the separation section 14′ for stripping volatiles from a hydrocracked stream. For example, the stripping column 190 may be in downstream communication with the hot bottoms line 154, the flash hot bottoms line 176, the cold bottoms line 160 and/or the cold flash bottoms line 182. In an aspect, the stripping column 190 may be a vessel that contains a cold stripping column 192 and a hot stripping column 194 with a wall that isolates each of the stripping columns 192, 194 from the other. The cold stripping column 192 may be in downstream communication with the hydrocracking reactor 140, the cold bottoms line 160 and, in an aspect, the flash cold bottoms line 182 for stripping the cold liquid stream. The hot stripping column 194 may be in downstream communication with the hydrocracking reactor 140 and the hot bottoms line 154 and, in an aspect, the flash hot bottoms line 176 for stripping a hot liquid stream which is hotter than the cold liquid stream. The hot liquid stream may be hotter than the cold liquid stream, by at least 25° C. and preferably at least 50° C.

The flash cold liquid stream comprising the hydrocracked stream in the flash cold bottoms line 176 may be heated and fed to the cold stripping column 192 at an inlet which may be in a top half of the column. The flash cold liquid stream which comprises the hydrocracked stream may be stripped of gases in the cold stripping column 192 with a cold stripping media which is an inert gas such as steam from a cold stripping media line 196 to provide a cold stripper gaseous stream of naphtha, hydrogen, hydrogen sulfide, steam and other gases in a cold stripper overhead line 198 and a liquid cold stripped stream in a cold stripper bottoms line 200. The cold stripper gaseous stream in the cold stripper overhead line 198 may be condensed and separated in a receiver 202. A stripper net overhead line 204 from the receiver 202 carries a net stripper gaseous stream for further recovery of LPG and hydrogen in a light material recovery unit. Unstabilized liquid naphtha from the bottoms of the receiver 202 may be split between a reflux portion refluxed to the top of the cold stripping column 192 and a liquid stripper overhead stream which may be transported in a condensed stripper overhead line 206 to further recovery or processing. A sour water stream may be collected from a boot of the overhead receiver 202.

The cold stripping column 192 may be operated with a bottoms temperature between about 149° C. (300° F.) and about 288° C. (550° F.), preferably no more than about 260° C. (500° F.), and an overhead pressure of about 0.35 MPa (gauge) (50 psig), preferably no less than about 0.50 MPa (gauge) (72 psig), to no more than about 2.0 MPa (gauge) (290 psig). The temperature in the overhead receiver 112 ranges from about 38° C. (100° F.) to about 66° C. (150° F.) and the pressure is essentially the same as in the overhead of the cold stripping column 192.

The cold stripped stream in the cold stripper bottoms line 200 may comprise predominantly naphtha and kerosene boiling materials. The cold stripped stream in line 200 may be heated and fed to the product fractionation column 210. The product fractionation column 210 may be in downstream communication with the hydrocracking reactor 140, the cold stripper bottoms line 200 of the cold stripping column 192 and the stripping column 190. In an aspect, the fractionation column 210 may comprise more than one fractionation column. The product fractionation column 210 may be in downstream communication with one, some or all of the hot separator 150, the cold separator 156, the hot flash drum 172 and the cold flash drum 178.

The flash hot liquid stream comprising a hydrocracked stream in the hot flash bottoms line 176 may be fed to the hot stripping column 194 near a top thereof. The flash hot liquid stream may be stripped in the hot stripping column 194 of gases with a hot stripping media which is an inert gas such as steam from a line 208 to provide a hot stripper overhead stream of naphtha, hydrogen, hydrogen sulfide, steam and other gases in a hot stripper overhead line 212 and a liquid hot stripped stream in a hot stripper bottoms line 214. The hot stripper overhead line 212 may be condensed and a portion refluxed to the hot stripping column 104. However, in the embodiment of FIG. 2, the hot stripper overhead stream in the hot stripper overhead line 212 from the overhead of the hot stripping column 194 may be fed into the cold stripping column 192 directly in an aspect without condensing or refluxing. The inlet for the cold flash bottoms line 182 carrying the flash cold liquid stream may be at a higher elevation than the inlet for the hot stripper overhead line 212. The hot stripping column 194 may be operated with a bottoms temperature between about 160° C. (320° F.) and about 360° C. (680° F.) and an overhead pressure of about 0.35 MPa (gauge) (50 psig), preferably no less than about 0.50 MPa (gauge) (72 psig), to about 2.0 MPa (gauge) (292 psig).

At least a portion of the hot stripped stream comprising a hydrocracked effluent stream in the hot stripped bottoms line 214 may be heated and fed to the product fractionation column 210. The product fractionation column 210 may be in downstream communication with the hot stripped bottoms line 214 of the hot stripping column 194. The hot stripped stream in line 214 may be at a hotter temperature than the cold stripped stream in line 200.

In an aspect, the hot stripped stream in the hot stripped bottoms line 214 may be heated and fed to a prefractionation separator 216 for separation into a vaporized hot stripped stream in a prefractionation overhead line 218 and a liquid hot stripped stream in a prefractionation bottoms line 220. The vaporous hot stripped stream may be fed to the product fractionation column 210 in the prefractionation overhead line 218 The liquid hot stripped stream may be heated in a fractionation furnace and fed to the product fractionation column 210 in the prefractionation bottoms line 220 at an elevation below the elevation at which the prefractionation overhead line 218 feeds the vaporized hot stripped stream to the product fractionation column 210.

The product fractionation column 210 may be in downstream communication with the cold stripping column 192 and the hot stripping column 194 and may comprise more than one fractionation column for separating stripped hydrocracked streams into product streams. The product fractionation column 210 may also be in downstream communication with said hot separator 150, the cold separator 156, hot flash drum 172 and the cold flash drum 178. The product fractionation column 210 may fractionate hydrocracked streams, the cold stripped stream, the vaporous hot stripped stream and the liquid hot stripped stream by means of an inert stripping stream fed from stripping line 234. The product streams from the product fractionation column 210 may include a net fractionated overhead stream comprising naphtha in a net overhead line 226, an optional heavy naphtha stream in line 228 from a side cut outlet, a kerosene stream carried in line 230 from a side cut outlet and a diesel liquid stream in diesel line 232 from a side cut outlet 232o.

An UCO stream boiling above the diesel cut point may be taken in a fractionator bottoms line 240 from a bottom of the product fractionation column 210. A portion or all of the UCO stream in the fractionator bottoms line 240 may be purged from the process, recycled to the hydrocracking reactor 140 or forwarded to a second stage hydrocracking reactor (not shown).

Product streams may also be stripped to remove light materials to meet product purity requirements. A fractionated overhead stream in an overhead line 248 may be condensed and separated in a receiver 250 with a portion of the condensed liquid being refluxed back to the product fractionation column 210. The net fractionated overhead stream in line 226 may be further processed or recovered as naphtha product. The product fractionation column 210 may be operated with a bottoms temperature between about 260° C. (500° F.) and about 385° C. (725° F.), preferably at no more than about 350° C. (650° F.), and at an overhead pressure between about 7 kPa (gauge) (1 psig) and about 69 kPa (gauge) (10 psig). A portion of the UCO stream in the atmospheric bottoms line 240 may be reboiled and returned to the product fractionation column 210 instead of adding an inert stripping media stream such as steam in line 234 to heat to the atmospheric fractionation column 210.

The diesel liquid stream in the diesel line 232 from the side cut outlet 232o may be stripped in a diesel stripper column 252 to meet product requirements and remove volatiles from the diesel stream. The diesel stripper column 252 may be in downstream communication with the side outlet 232o of the product fractionation column 210. The diesel stripper column 252 preferably uses a reboiler 224 instead of using stripping media such as steam to strip the diesel liquid stream with a reboiled stream in a reboiler line 246. The reboiler 224 receives a diesel liquid stream in line 232 from the side outlet 232o of the product fractionation column 210. A stripped diesel stream exits the diesel stripper column 252 in line 244 and a portion of the diesel stream is reboiled in reboil line 246 in a reboiler 224 and returned to the diesel stripper column. Stripping media which is an inert gas such as steam from a stripping media line is preferably not used in the diesel stripper column 252 to avoid adding water to the diesel stripping column and the stripped diesel stream in a stripped bottoms line 70′. The reboiled stream in reboil line 246 may comprise no more than about 1 wt % water. The reboiler 224 may be a fired heater or a heat exchanger. Volatiles and stripping media leave the overhead of the diesel striper in line 254 and are returned to the product fractionation column 210. Similar stripping may be performed on the kerosene stream 230 and the heavy naphtha stream in line 228. Alternatively, the kerosene stream may be processed with the diesel stream in line 232.

The diesel stripper column 252 may be operated with a bottoms temperature between about 160° C. (320° F.) and about 370° C. (700° F.), and an overhead pressure of about 0.35 MPa (gauge) (50 psig), preferably about 0.70 MPa (gauge) (100 psig), to about 2.0 MPa (gauge) (300 psig). The temperature in the overhead of the diesel stripping column ranges from about 200° C. (400° F.) to about 300° C. (570° F.) and the pressure is essentially the same as in the product fractionation column 210.

The stripped hydroprocessed stream in the stripped bottoms line 70′ comprises predominantly diesel range boiling material. The stripped hydroprocessed stream may have an end point between about 343° C. (650° F.) and about 399° C. (750° F.) and may have an IBP in the range of between about 132° C. (270° F.) and about 210° C. (410° F.). As previously explained, a stripped kerosene stream may be taken in the stripped bottoms line 70′ instead of or with diesel.

During the colder months, the cold flow properties of the distillate in the stripped hydroprocessed stream in the stripped bottoms line 70′ may need improvement to meet winter specifications. Consequently, the distillate is treated in the hydroisomerization unit 110′. A valve on the stripped bottoms line 70′ is opened, so the stripped hydroprocessed liquid stream in the stripped bottoms line 70′ may be passed to the hydroisomerization unit 110′. The stripped hydroprocessed liquid stream is supplemented with a hydroisomerization hydrogen stream in a hydroisomerization hydrogen line 76, heat exchanged with hydroisomerized effluent in a hydroisomerized effluent line 78 and fed to a hydroisomerization reactor 80 in the hydroisomerization unit 110 as explained with respect to the stripped hydroprocessed stream in the stripped bottoms line 70 of FIG. 1. The operation of the hydroisomerization unit 110 is the same as described for FIG. 1.

### Specific Embodiments

While the following is described in conjunction with specific embodiments, it will be understood that this description is intended to illustrate and not limit the scope of the preceding description and the appended claims.

A first embodiment of the invention is a process comprising hydroprocessing a hydrocarbon feed stream in a hydroprocessing reactor to provide a hydroprocessing effluent stream at a hydroprocessing pressure; separating the hydroprocessing effluent stream in a separator to provide a gaseous stream and a liquid stream; stripping light gases from the liquid stream to provide a stripper off gas stream and a stripped hydroprocessed stream; adding hydrogen to the stripped hydroprocessed stream; and hydroisomerizing the stripped hydroprocessed stream over a hydroisomerization catalyst at a hydroisomerization pressure that is less than the hydroprocessing pressure. An embodiment of the invention is one, any or all of prior embodiments in this paragraph up through the first embodiment in this paragraph wherein the stripped hydroprocessed stream has an end point between about 343° C. (650° F.) and about 399° C. (750° F.). An embodiment of the invention is one, any or all of prior embodiments in this paragraph up through the first embodiment in this paragraph wherein the stripped hydroprocessed stream has an IBP in the range of between about 132° C. (270° F.) and about 210° C. (410° F.). An embodiment of the invention is one, any or all of prior embodiments in this paragraph up through the first embodiment in this paragraph wherein the hydroisomerization pressure is less than about 2.7 MPa (gauge) (400 psig). An embodiment of the invention is one, any or all of prior embodiments in this paragraph up through the first embodiment in this paragraph wherein the hydroprocessing pressure is at least about 4.1 MPa (gauge) (600 psig). An embodiment of the invention is one, any or all of prior embodiments in this paragraph up through the first embodiment in this paragraph further comprising stripping the liquid stream with a reboiled stream. An embodiment of the invention is one, any or all of prior embodiments in this paragraph up through the first embodiment in this paragraph wherein the reboiled stream comprises no more than about 1 wt % water. An embodiment of the invention is one, any or all of prior embodiments in this paragraph up through the first embodiment in this paragraph further comprising stripping the liquid stream in a stripper column that receives the liquid stream from a side outlet of the product fractionation column. An embodiment of the invention is one, any or all of prior embodiments in this paragraph up through the first embodiment in this paragraph further comprising stripping the liquid stream in a stripper column in direct communication with a separator. An embodiment of the invention is one, any or all of prior embodiments in this paragraph up through the first embodiment in this paragraph further comprising bypassing the stripped hydroprocessed stream around a hydroisomerization reactor and terminating the hydroisomerization step. An embodiment of the invention is one, any or all of prior embodiments in this paragraph up through the first embodiment in this paragraph further comprising separating a hydroisomerized stream into a naphtha stream and a diesel stream.

A second embodiment of the invention is a process comprising hydroprocessing a hydrocarbon feed stream in a hydroprocessing reactor to provide a hydroprocessing effluent stream at a hydroprocessing pressure; separating the hydroprocessing effluent stream in a separator to provide a gaseous stream and a liquid stream; stripping light gases from the liquid stream with a reboiled stream to provide a stripper off gas stream and a stripped hydroprocessed stream; adding hydrogen to the stripped hydroprocessed stream; and hydroisomerizing the stripped hydroprocessed stream over a hydroisomerization catalyst at a hydroisomerization pressure that is less than the hydroprocessing pressure. An embodiment of the invention is one, any or all of prior embodiments in this paragraph up through the second embodiment in this paragraph wherein the hydroisomerization pressure is less than about 2.7 MPa (gauge) (400 psig) and the hydroprocessing pressure is at least about 4.1 MPa (gauge) (600 psig). An embodiment of the invention is one, any or all of prior embodiments in this paragraph up through the second embodiment in this paragraph wherein the reboiled stream comprises no more than about 1 wt % water. An embodiment of the invention is one, any or all of prior embodiments in this paragraph up through the second embodiment in this paragraph further comprising stripping the liquid stream in a side stripper column that receives a product stream from a product fractionation column. An embodiment of the invention is one, any or all of prior embodiments in this paragraph up through the second embodiment in this paragraph further comprising stripping the liquid stream in a stripper column in direct communication with a separator. A apparatus comprising a hydroprocessing reactor; a separator for separating a hydroprocessed effluent from the hydroprocessing reactor into a hydroprocessed liquid stream; a stripper column for stripping light gasses from the hydroprocessed liquid stream; a hydroisomerization reactor in downstream communication with the stripper column. An embodiment of the invention is one, any or all of prior embodiments in this paragraph up through the second embodiment in this paragraph wherein the hydroprocessing reactor is a hydrocracking reactor and further comprising a product fractionation column in downstream communication with the separator and the stripper is in downstream communication with a side outlet of the product fractionation column. An embodiment of the invention is one, any or all of prior embodiments in this paragraph up through the second embodiment in this paragraph wherein the hydroprocessing reactor is a hydrotreating reactor and the stripping column is in direct, downstream communication with the separator. An embodiment of the invention is one, any or all of prior embodiments in this paragraph up through the second embodiment in this paragraph further comprising a bypass line in downstream communication with the stripper column but out of communication with the hydroisomerization reactor for bypassing a stripped hydroprocessed liquid stream around the hydroisomerization reactor.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A hydroprocessing process comprising:
hydroprocessing a hydrocarbon feed stream in a hydroprocessing reactor to provide a hydroprocessing effluent stream at a hydroprocessing pressure;
separating said hydroprocessing effluent stream in a separator to provide a gaseous stream and a liquid stream;
stripping light gases from said liquid stream to provide a stripper off gas stream and a stripped hydroprocessed stream;
adding hydrogen to said stripped hydroprocessed stream; and
hydroisomerizing said stripped hydroprocessed stream over a hydroisomerization catalyst at a hydroisomerization pressure that is less than the hydroprocessing pressure.

2. The process of claim 1 wherein said stripped hydroprocessed stream has an end point between about 343° C. (650° F.) and about 399° C. (750° F.).

3. The process of claim 2 wherein said stripped hydroprocessed stream has an IBP in the range of between about 132° C. (270° F.) and about 210° C. (410° F.)

4. The process of claim 1 wherein said hydroisomerization pressure is less than about 2.7 MPa (gauge) (400 psig).

5. The process of claim 1 wherein said hydroprocessing pressure is at least about 4.1 MPa (gauge) (600 psig).

6. The process of claim 1 further comprising stripping said liquid stream with a reboiled stream.

7. The process of claim 6 wherein said reboiled stream comprises no more than about 1 wt % water.

8. The process of claim 1 further comprising stripping said liquid stream in a stripper column that receives the liquid stream from a side outlet of the product fractionation column.

9. The process of claim 1 further comprising stripping said liquid stream in a stripper column in direct communication with a separator.

10. The process of claim 1 further comprising bypassing said stripped hydroprocessed stream around a hydroisomerization reactor and terminating said hydroisomerization step.

11. The process of claim 1 further comprising separating a hydroisomerized stream into a naphtha stream and a diesel stream.

12. A hydroprocessing process comprising:
hydroprocessing a hydrocarbon feed stream in a hydroprocessing reactor to provide a hydroprocessing effluent stream at a hydroprocessing pressure;
separating said hydroprocessing effluent stream in a separator to provide a gaseous stream and a liquid stream;
stripping light gases from said liquid stream with a reboiled stream to provide a stripper off gas stream and a stripped hydroprocessed stream;
adding hydrogen to said stripped hydroprocessed stream; and
hydroisomerizing said stripped hydroprocessed stream over a hydroisomerization catalyst at a hydroisomerization pressure that is less than the hydroprocessing pressure.

13. The process of claim 12 wherein said hydroisomerization pressure is less than about 2.7 MPa (gauge) (400 psig) and said hydroprocessing pressure is at least about 4.1 MPa (gauge) (600 psig).

14. The process of claim 13 wherein said reboiled stream comprises no more than about 1 wt % water.

15. The process of claim 12 further comprising stripping said liquid stream in a side stripper column that receives a product stream from a product fractionation column.

16. The process of claim 12 further comprising stripping said liquid stream in a stripper column in direct communication with a separator.

17. A hydroprocessing apparatus comprising:
a hydroprocessing reactor;
a separator for separating a hydroprocessed effluent from said hydroprocessing reactor into a hydroprocessed liquid stream;
a stripper column for stripping light gasses from said hydroprocessed liquid stream; and
a hydroisomerization reactor in downstream communication with said stripper column.

18. The hydroprocessing apparatus of claim 17 wherein said hydroprocessing reactor is a hydrocracking reactor and further comprising a product fractionation column in downstream communication with said separator and said stripper is in downstream communication with a side outlet of said product fractionation column.

19. The hydroprocessing apparatus of claim 17 wherein said hydroprocessing reactor is a hydrotreating reactor and said stripping column is in direct, downstream communication with said separator.

20. The hydroprocessing apparatus of claim 17 further comprising a bypass line in downstream communication with said stripper column but out of communication with said hydroisomerization reactor for bypassing a stripped hydroprocessed liquid stream around the hydroisomerization reactor.
